(19) United States
(12) Patent Application Publication
(10) Pub. No.: US 2023/0158001 A1
(43) Pub. Date: May 25, 2023

(54) PHARMACEUTICAL COMPOSITION COMPRISING BENZIMIDAZOLE DERIVATIVE COMPOUND

(71) Applicant: HK INNO.N CORPORATION, Cheongju-si Chungcheongbuk-do (KR)

(72) Inventors: Seokuee KIM, Gyeonggi-do (KR); Yeong Hyeon JO, Seoul (KR); Eun Jee PARK, Seoul (KR); Bong Tae KIM, Gyeonggi-do (KR); Heehyun KIM, Seoul (KR); Minja KANG, Chungcheongbuk-do (KR); Geun Seog SONG, Seoul (KR); Naree SHIN, Incheon (KR); Eun Ji KIM, Seoul (KR); Eunbi CHO, Seoul (KR)

(21) Appl. No.: 17/918,386

(22) PCT Filed: Apr. 13, 2021
(86) PCT No.: PCT/IB2021/053034

§ 371 Date: Jan. 27, 2023

(30) Foreign Application Priority Data
Apr. 13, 2020 (KR) 10-2020-0044864

(51) Int. Cl. A61K 31/4184; A61K 45/06; A61K 31/19; A61P 29/00

(52) U.S. Cl. CPC A61K 31/4184; A61K 45/06; A61K 31/19; A61P 29/00

(57) ABSTRACT

The present invention relates to a pharmaceutical composition for preventing peptic ulcer and/or recurrence thereof containing a benzimidazole derivative. The pharmaceutical composition of the present invention may effectively prevent peptic ulcer and/or recurrence thereof, which may be caused by administration of a non-steroidal anti-inflammatory drug, for a long period of time without side effects.

### TECHNICAL FIELD

The present invention relates to a pharmaceutical composition for inhibiting the occurrence and/or recurrence of peptic ulcer containing a benzimidazole derivative compound.

### BACKGROUND ART

Peptic ulcer is one of the most common diseases in clinic, and causes persistent pain and interferes with the patient's daily life, and if not treated, it may cause serious complications such as bleeding and perforation or may recur frequently.

With the improvement of living standards and increasing interest in health, upper gastrointestinal endoscopy has been widely performed, and with the development of endoscopy equipment and diagnostic technology, there have been many advances in the detection, diagnosis, and treatment of upper gastrointestinal diseases.

However, as a use of non-steroidal anti-inflammatory drugs (NSAIDs) has increased due to the increase in musculoskeletal and cardiovascular diseases with the aging of the population, the occurrence of peptic ulcer and its complications such as bleeding has not yet decreased. In particular, even if peptic ulcer has been treated, its recurrence occurs frequently due to the increased use of non-steroidal anti-inflammatory drugs. However, even when the gastric acid secretion inhibitor used for the treatment of previous peptic ulcer is applied, the recurrent peptic ulcer tends to not be treated well, and if recurrence is repeated, complications such as bleeding may occur more frequently.

Therefore, it is important to safely and effectively prevent peptic ulcer and/or recurrence thereof without side effects even when taking non-steroidal anti-inflammatory drugs after treatment of peptic ulcer.

### PRIOR ART DOCUMENTS

### Patent Documents

Korean Patent No. 10-1088247

### DISCLOSURE

### Technical Problem

An object of the present invention is to provide a pharmaceutical composition for preventing peptic ulcer and/or recurrence thereof, the pharmaceutical composition containing tegoprazan, which is a compound represented by the following Formula 1, or a pharmaceutically acceptable salt thereof.

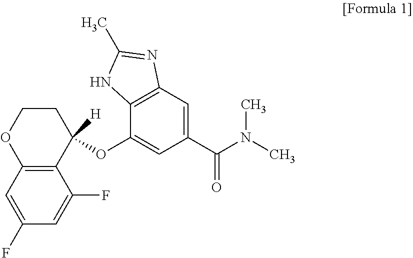 [SMILES: Cc1nc2cc(C(=O)N(C)C)cc(O[C@H]3CCOc4cc(F)cc(F)c43)c2[nH]1]

### Technical Solution

The present invention provides a pharmaceutical composition for preventing peptic ulcer and/or recurrence thereof, the pharmaceutical composition containing, as an active ingredient, tegoprazan, which is a compound represented by the following Formula 1, an optical isomer thereof, a pharmaceutically acceptable salt thereof, a hydrate or solvate thereof, or a mixture thereof, wherein the pharmaceutical composition may prevent occurrence and/or recurrence of peptic ulcer caused by administration of a non-steroidal anti-inflammatory drug.

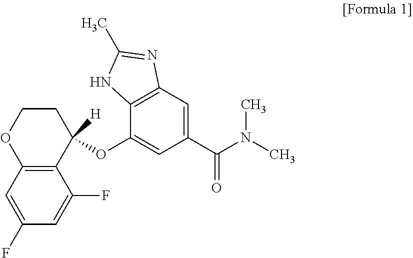 [SMILES: Cc1nc2cc(C(=O)N(C)C)cc(O[C@H]3CCOc4cc(F)cc(F)c43)c2[nH]1]

The pharmaceutical composition of the present invention may effectively inhibit peptic ulcer and/or recurrence thereof. Specifically, the pharmaceutical composition of the present invention may safely and effectively prevent peptic ulcer and/or recurrence thereof caused by administration of non-steroidal anti-inflammatory drug in a subject, who takes the non-steroidal anti-inflammatory drug, for a long period of time without side effects.

Non-steroidal anti-inflammatory drugs are administered for a long period of time in many cases, and particularly, the duration and frequency of administration of non-steroidal anti-inflammatory drugs have continued to increase due to the aging of the population. However, non-steroidal anti-inflammatory drugs may cause peptic ulcer, and especially in the case of subjects who already experienced peptic ulcer but have been treated, peptic ulcer is more likely to recur due to administration of non-steroidal anti-inflammatory drugs, and symptoms thereof may be more severe than previously experienced.

The pharmaceutical composition of the present invention, which contains, as an active ingredient, tegoprazan, an optical isomer thereof, a pharmaceutically acceptable salt thereof, a hydrate or solvate thereof, or a mixture thereof, has high safety, may be administered for a long period of time without side effects, and may significantly effectively prevent, for a long period of time, peptic ulcer from occurring and/or recurring due to administration of non-steroidal anti-inflammatory drugs.

The pharmaceutical composition of the present invention may significantly effectively prevent peptic ulcer from occurring and/or recurring due to administration of non-steroidal anti-inflammatory drugs, without being affected by CYP2C19 genetic polymorphism and without side effects.

In the present invention, tegoprazan which is a compound represented by the Formula 1 is also referred to as “(S)-4-(5,7-difluorochroman-4-yloxy)-N,N,2-trimethyl-1H-benzo[d]imidazole-6-carboxamide”.

When tegoprazan is referred to in the present specification, it may refer to tegoprazan, an optical isomer thereof, a pharmaceutically acceptable salt thereof, a hydrate or solvate thereof, or a mixture thereof. Therefore, in the present specification, the pharmaceutical composition containing tegoprazan may refer to a composition containing tegoprazan, an optical isomer thereof, a pharmaceutically acceptable salt thereof, a hydrate or solvate thereof, or a mixture thereof.

In the present invention, peptic ulcer is a defect in the gastrointestinal tract mucosa. Generally, peptic ulcer refers to a case in which a histologically necrotic mucosa defect extends through the muscularis mucosa to the submucosa or the muscularis propria. Peptic ulcer may include ulcers occurring in the stomach and/or duodenum. In the present invention, the term “peptic ulcer” may be used interchangeably with the term “gastric ulcer” and/or “duodenal ulcer”, the term “gastric and/or duodenal ulcer”, and the term “gastric/duodenal ulcer”.

In the present invention, the term “subject” may refer to mammals, including humans, and specifically, may refer to humans. In the present invention, the subject may be a person at risk of developing peptic ulcer (gastric ulcer and/or duodenal ulcer) associated with a non-steroidal anti-inflammatory drug. For example, the subject may be a person who needs to continuously take a non-steroidal anti-inflammatory drug.

In the examples of the present invention, the subject may be a person who experienced peptic ulcer at least once before administration of the pharmaceutical composition, but has been treated. Specifically, the subject may be a person who experienced peptic ulcer at least once before administration of the pharmaceutical composition, but has been treated and is at a high risk of developing peptic ulcer associated with a non-steroidal anti-inflammatory drug. In the present invention, the onset or treatment of peptic ulcer may be determined through endoscopy.

In examples of the present invention, when the presence of an ulcer and/or a bleeding lesion in the upper gastrointestinal tract (such as stomach or duodenum) is confirmed by endoscopy, it may be determined that peptic ulcer has occurred. For example, when active stage (A1 and/or A2) or healing stage (H1 and/or H2) according to the Sakita-Miwa classification is diagnosed through endoscopy, it may be determined that peptic ulcer has occurred.

In examples of the present invention, when the presence of an ulcer and/or a bleeding lesion in the gastrointestinal tract (such as stomach or duodenum) is not confirmed by endoscopy or only an ulcer scar is confirmed through endoscopy, it may be determined that peptic ulcer did not develop. For example, when scarring stage (S1 and/or S2) according to the Sakita-Miwa classification method or the presence of an ulcer and/or a bleeding lesion is not confirmed by endoscopy, it may be determined that peptic ulcer did not develop.

In the present invention, the term “ulcer scar” may refer to a faded scar in which tissues such as mucous membranes and muscle layers injured by the ulcer are recovered and the regenerated epithelium remains red only and shows a red scar or a small faded spot in the center.

In examples of the present invention, when there are heartburn, gastric acid reflux, and/or epigastric pain or discomfort, it may be determined that peptic ulcer has occurred.

In examples of the present invention, when symptoms of heartburn, gastric acid reflux, and epigastric pain or discomfort do not appear, it may be determined that peptic ulcer did not occur or has been treated.

In the present invention, the expression “preventing peptic ulcer and/or recurrence thereof” may include preventing peptic ulcer and/or recurrence thereof or inhibiting or delaying peptic ulcer and/or recurrence thereof. For example, the expression “preventing peptic ulcer” may include preventing, inhibiting, or delaying the onset of peptic ulcer in a subject, and the expression “preventing recurrence of peptic ulcer” may include preventing, inhibiting or delaying the onset of peptic ulcer in a subject who experienced peptic ulcer, but has been treated. The subject may be a person at risk of developing peptic ulcer associated with a non-steroidal anti-inflammatory drug. Specifically, the subject may be a person who needs to continuously take a non-steroidal anti-inflammatory drug.

The present invention provides a pharmaceutical composition for preventing peptic ulcer and/or recurrence thereof, the pharmaceutical composition containing tegoprazan, an optical isomer thereof, a pharmaceutically acceptable salt thereof, a hydrate or solvate thereof, or a mixture thereof, wherein the peptic ulcer and/or recurrence thereof may be caused by administration of a non-steroidal anti-inflammatory drug.

In examples of the present invention, the pharmaceutical composition is used to prevent peptic ulcer and/or recurrence thereof by administering to a subject a pharmaceutical composition containing tegoprazan, an optical isomer thereof, a pharmaceutically acceptable salt thereof, a hydrate or solvate thereof, or a mixture thereof in an amount of 25 mg to 50 mg as tegoprazan (a free base form), wherein the peptic ulcer and/or recurrence thereof may be caused by administration of a non-steroidal anti-inflammatory drug. Specifically, the pharmaceutical composition may contain tegoprazan, an optical isomer thereof, a pharmaceutically acceptable salt thereof, a hydrate or solvate thereof, or a mixture thereof in an amount of 25 mg to 50 mg as tegoprazan (a free base form). In the present invention, the “an amount of 25 mg to 50 mg as tegoprazan (a free base form)” may be used interchangeably with “an amount of 25 mg to 50 mg based on tegoprazan (a free base form)”.

In examples of the present invention, the subject may be a person who experienced peptic ulcer at least once before administration of the pharmaceutical composition. Specifically, the subject may be a person who was diagnosed with an ulcer in the gastrointestinal tract such as the stomach and/or duodenum at least once, twice, or twice or more through endoscopy before administration of the pharmaceutical composition. More specifically, the subject may be a person who was diagnosed with a gastrointestinal ulcer such as a gastric and/or duodenal ulcer, at least once, at least two times, or at least three times through endoscopy within 5 years before administration of the pharmaceutical composition. For example, the subject may be a person when was diagnosed with an active stage (A1 and/or A2) or healing stage (H1 and/or H2) ulcer according to the Sakita-Miwa classification through endoscopy within 5 years before administration of the pharmaceutical composition or experienced heartburn, gastric acid reflux, and/or epigastric pain or discomfort due to an ulcer within 5 years before administration of the pharmaceutical composition.

In examples of the present invention, the degree, number of onsets, duration and/or cause of the peptic ulcer experienced by the subject before administration of the pharmaceutical composition may be diverse. For example, the peptic ulcer experienced by the subject before administration of the pharmaceutical composition may be caused by or unrelated to administration of a non-steroidal anti-inflammatory drug.

In examples of the present invention, the subject may be a person whose peptic ulcer has been treated after the onset of the peptic ulcer and before administration of the pharmaceutical composition. Specifically, the subject had ever developed peptic ulcer, but may be a person who has been determined by endoscopy before administration of the pharmaceutical composition to be in a state in which an ulcer and a bleeding lesion do not exist in the digestive tract such as the stomach or duodenum or only an ulcer scar is present. More specifically, the subject may be in a state in which an ulcer and a bleeding lesion does not exist in the digestive tract such as the stomach and/or duodenum or only an ulcer scar is present, as determined by endoscopy before administration of the pharmaceutical composition, specifically within 4 weeks, for example, 2 weeks, before administration of the pharmaceutical composition, after diagnosis of peptic ulcer, or the subject may be a person who does not exhibit symptoms such as heartburn, acid reflux, and epigastric pain or discomfort before administration of the pharmaceutical composition. For example, as a result of confirming through endoscopy, before administration of the pharmaceutical composition, the subject may be in a state in which an ulcer and a bleeding lesion do not exist or in a scarring stage (S1 and/or S2) according to the Sakita-Miwa classification, or may be a person who does not exhibit symptoms such as heartburn, acid reflux, and epigastric pain or discomfort.

The pharmaceutical composition of the present invention may effectively prevent recurrence of peptic ulcer regardless of the degree, duration, number of onsets, or cause of the peptic ulcer experienced by the subject before administration of the pharmaceutical composition. For example, the pharmaceutical composition of the present invention may effectively prevent, delay or inhibit recurrence of peptic ulcer regardless of the severity, duration, number of onsets, or cause of the peptic ulcer experienced by a subject before administration of the pharmaceutical composition.

In examples of the present invention, the subject may be a person having an underlying disease. For example, the subject may be a person having a musculoskeletal disorder as an underlying disease.

In examples of the present invention, the pharmaceutical composition may be for co-administration with a non-steroidal anti-inflammatory drug. Specifically, the pharmaceutical composition may be co-administered with a non-steroidal anti-inflammatory drug. The pharmaceutical composition of the present invention may be co-administered with a non-steroidal anti-inflammatory drug to effectively and safely prevent, for a long period of time, peptic ulcer from occurring and/or recurring due to administration of the non-steroidal anti-inflammatory drug. In addition, not only in the case in which a subject has no underlying disease, but also in a case in which a subject has an underlying disease, the pharmaceutical composition of the present invention may effectively and safely prevent peptic ulcer or recurrence thereof for a long period of time without side effects.

In addition, even when the pharmaceutical composition of the present invention is co-administered with a non-steroidal anti-inflammatory drug, the pharmacokinetic characteristics of each of the composition and the non-steroidal anti-inflammatory drug may not be affected. For example, even when the pharmaceutical composition of the present invention is co-administered with a non-steroidal anti-inflammatory drug, it may exhibit a sufficient pharmacological effect without deteriorating the efficacy of each of the active ingredient tegoprazan of the pharmaceutical composition and the non-steroidal anti-inflammatory drug.

In examples of the present invention, the pharmaceutical composition and non-steroidal anti-inflammatory drug may be each independently formulated in separate unit dosage forms and co-administered. Alternatively, in examples of the present invention, the pharmaceutical composition may also be formulated in a single unit dosage form containing a non-steroidal anti-inflammatory drug. Specifically, the pharmaceutical composition and the non-steroidal anti-inflammatory drug may be formulated in combination (complex) formulation and co-administered.

In examples of the present invention, the unit dosage form may be a tablet or a capsule. For example, when the unit dosage form is a tablet, the tablet may be prepared by tableting a mixture or granules containing the pharmaceutical composition, the non-steroidal anti-inflammatory drug, or a mixture thereof. For example, when the unit dosage form is a capsule, the capsule may be a capsule filled with a granule, pellet or tablet containing the pharmaceutical composition, the non-steroidal anti-inflammatory drug, or a mixture thereof, and in this case, the tablet may be a mini-tablet.

In the present invention, the term “unit dosage form” may be used interchangeably with the term “unit formulation”.

In examples of the present invention, the subject may be a person who takes a non-steroidal anti-inflammatory drug for 4 weeks or more, or 8 weeks or more, specifically 10 weeks or more, more specifically 12 weeks or more, even more specifically 24 weeks or more.

In examples of the present invention, the pharmaceutical composition and the non-steroidal anti-inflammatory drug may be administered to a subject simultaneously, or sequentially, or separately with a time lag of less than 1 day. Specifically, the pharmaceutical composition may be administered simultaneously with the non-steroidal anti-inflammatory drug, or may be administered continuously before or after administration of the non-steroidal anti-inflammatory drug.

In examples of the present invention, in case that the pharmaceutical composition is to be administered simultaneously with a non-steroidal anti-inflammatory drug, the pharmaceutical composition and the non-steroidal anti-inflammatory drug may be formulated in separate unit dosage forms and administered simultaneously, or the pharmaceutical composition and the non-steroidal anti-inflammatory drug may be formulated in a single unit dosage form and administered simultaneously.

In examples of the present invention, the pharmaceutical composition and the non-steroidal anti-inflammatory drug may be co-administered for 4 weeks or more, or 8 weeks or more, specifically 10 weeks or more, more specifically 12 weeks or more. For example, the pharmaceutical composition may be co-administered with the non-steroidal anti-inflammatory drug for 12 to 24 weeks.

As the composition of the present invention is co-administered with the non-steroidal anti-inflammatory drug, it may effectively prevent peptic ulcer and/or recurrence thereof caused by the non-steroidal anti-inflammatory drug.

In examples of the present invention, the subject may be a person who takes a non-steroidal anti-inflammatory drug before administration of the pharmaceutical composition. Specifically, the subject may be a person who takes a non-steroidal anti-inflammatory drug from 4 weeks before administration of the pharmaceutical composition. For example, a non-steroidal anti-inflammatory drug may be administered to the subject from 4 weeks before administration of the pharmaceutical composition, and then the pharmaceutical composition and the non-steroidal anti-inflammatory drug may be co-administered for 4 to 24 weeks.

In examples of the present invention, a non-steroidal anti-inflammatory drug may be orally administered to the subject.

In examples of the present invention, the reason for administering the non-steroidal anti-inflammatory drug to the subject is not particularly limited, and regardless of the reason for administering the non-steroidal anti-inflammatory drug, the composition of the present invention effectively prevent peptic ulcer caused by the non-steroidal anti-inflammatory drug. For example, the subject may be a person to whom a non-steroidal anti-inflammatory drug is administered for pain control, specifically a person to whom a non-steroidal anti-inflammatory drug is administered to control the pain associated with chronic musculoskeletal disease, more specifically a person to whom a non-steroidal anti-inflammatory drug is administered to control the pain associated with arthritis such as osteoarthritis or rheumatoid arthritis.

In examples of the present invention, the non-steroidal anti-inflammatory drug may be a non-selective non-steroidal anti-inflammatory drug (non-selective NSAID), a selective or non-selective Cox-2 inhibitor (Cox-2 inhibitor), aspirin, or a mixture thereof. Specifically, the non-steroidal anti-inflammatory drug may be selected from among salicylates, propionic acid derivatives, acetic acid derivatives, enolic acid (oxicam) derivatives, anthranilic acid derivatives (fenamates), selective Cox-2 inhibitors (coxibs), sulfonanilides, Clonixin, or mixtures thereof. More specifically, the non-steroidal anti-inflammatory drug may comprise one or more selected from the group consisting of propionic acid derivatives, acetic acid derivatives, and selective Cox-2 inhibitors (coxibs). More specifically, the non-steroidal anti-inflammatory drug may be selected from among aceclofenac, acemetacin, alminoprofen, amfenac, apazone, aspirin, bromfenac, bufexamac, celecoxib, choline salicylate, cinnoxicam, clonixin, dexibuprofen, dexketoprofen, diclofenac, diflunisal, emorfazone, etodolac, etoricoxib, ethenzamide, felbinac, fenoprofen, flufenamic acid, flurbiprofen, ibuprofen, imidazole salicylate, indomethacin, isopropylantipyrine, ketoprofen, ketorolac, lornoxicam, loxoprofen, meclofenamate, meloxicam, mefenamic acid, morniflumate, nabumetone, naproxen, nefopam, nimesulide, oxaprozin, oxyphenbutazone, pelubiprofen, phenylbutazone, piroxicam, pranoprofen, proglumetacin, rofecoxib, salsalate, salicylate, sulindac, talniflumate, tenoxicam, tiaprofenic acid, tolfenamic acid, tolmetin, valdecoxib, zaltoprofen, salicylic acid, meloxicam, isoxicam, droxicam, flufenamic acid, tolfenamic acid, lumiracoxib, firocoxib, parecoxib, pharmaceutically acceptable salts thereof, or mixtures thereof. In examples of the present invention, the non-steroidal anti-inflammatory drug may comprise, but is not limited to, at least one selected from the group consisting of naproxen, aceclofenac, and celecoxib.

In examples of the present invention, the expression “preventing peptic ulcer and/or recurrence thereof” means that a state in which an ulcer or a bleeding lesion does not exist is maintained for a certain period of time or continuously during continuous administration or after completion of administration of a non-steroidal anti-inflammatory drug. Specifically, the expression “preventing peptic ulcer and/or recurrence thereof” means that a state, in which the presence of an ulcer and a bleeding lesion in the digestive tract is not confirmed by endoscopy or only an ulcer scar is observed by endoscopy, is maintained for a certain period of time or continuously during continuous administration or after completion of administration of a non-steroidal anti-inflammatory drug. For example, the expression “preventing peptic ulcer and/or recurrence thereof” means that either a state in which the presence of an ulcer and a bleeding lesion in the digestive tract is not confirmed by endoscopy or a state classified as scarring stage (S1 and/or S2) according to the Sakita-Miwa classification is maintained for a certain period of time or continuously during continuous administration or after completion of administration of a non-steroidal anti-inflammatory drug.

In examples of the present invention, the expression “preventing peptic ulcer and/or recurrence thereof” means that a state in which symptoms caused by peptic ulcer do not appear is maintained for a certain period of time or continuously during continuous administration or after completion of administration of a non-steroidal anti-inflammatory drug. For example, the expression “preventing peptic ulcer and/or recurrence thereof” means that a state in which heartburn, gastric acid reflux, or epigastric pain or discomfort resulting from peptic ulcer does not appear is maintained for a certain period of time or continuously during continuous administration or after completion of administration of a non-steroidal anti-inflammatory drug.

The present invention provides a pharmaceutical composition for preventing gastric ulcer or duodenal ulcer and/or recurrence thereof, the pharmaceutical composition containing, as an active ingredient, tegoprazan, an optical isomer thereof, a pharmaceutically acceptable salt thereof, a hydrate or solvate thereof, or a mixture thereof, wherein the gastric ulcer or duodenal ulcer and/or recurrence may be caused by administration of a non-steroidal anti-inflammatory drug.

In examples of the present invention, the pharmaceutical composition may contain tegoprazan, an optical isomer thereof, a pharmaceutically acceptable salt thereof, a hydrate or solvate thereof, or a mixture thereof in an amount of 25 mg to 50 mg as tegoprazan (a free base form). Specifically, the pharmaceutical composition may contain tegoprazan or a pharmaceutically acceptable salt thereof in an amount of 25 mg or 50 mg as tegoprazan.

In examples of the present invention, the pharmaceutical composition may be administered once a day. Specifically, the tegoprazan may be administered once a day for 4 weeks to 24 weeks, specifically once a day for 4 weeks to 12 weeks. Specifically, the pharmaceutical composition containing tegoprazan or a pharmaceutically acceptable salt thereof in an amount of 25 mg to 50 mg as tegoprazan may be administered once a day for 4 weeks to 24 weeks, more specifically once a day for 4 weeks to 12 weeks.

In examples of the present invention, the pharmaceutical composition may be formulated in a unit dosage form, and in case that it is formulated in a unit dosage form such as a tablet or a capsule, the unit dosage form may be administered once a day regardless of diet. Specifically, the pharmaceutical composition containing tegoprazan, an optical isomer thereof, a pharmaceutically acceptable salt thereof, a hydrate or solvate thereof, or a mixture thereof in an amount of 25 mg to 50 mg as tegoprazan (a free base form) may be formulated in a unit dosage form.

In examples of the present invention, the pharmaceutical composition may be formulated in a unit dosage form separate from a non-steroidal anti-inflammatory drug. In this case, the pharmaceutical composition and the non-steroidal anti-inflammatory drug may be administered to a subject simultaneously, or continuously, or separately with a time lag of less than 1 day. Specifically, the pharmaceutical composition may be administered simultaneously with the non-steroidal anti-inflammatory drug, or may be continuously administered before or after administration of the non-steroidal anti-inflammatory drug.

In examples of the present invention, the pharmaceutical composition may be formulated in a single unit dosage form containing the non-steroidal anti-inflammatory drug and administered to a subject at the same time.

In case that the pharmaceutical composition is formulated in a unit dosage form such as a tablet or a capsule, the unit dosage form may be administered once a day, and may be administered regardless of diet. More specifically, the pharmaceutical composition containing tegoprazan, an optical isomer thereof, a pharmaceutically acceptable salt thereof, a hydrate or solvate thereof, or a mixture thereof in an amount of 25 mg or 50 mg as tegoprazan (a free base form) may be formulated in a unit dosage form, and in case that the pharmaceutical composition is formulated in a unit dosage form such as a tablet or a capsule, the unit dosage form may be administered once a day, and may be administered regardless of diet.

In the examples of the present invention, the term “pharmaceutically acceptable salt” refers to a salt formed with any inorganic or organic acid or base that does not cause serious irritation to a subject and does not impair the biological activity and physical properties of the compound. As the salt, a salt commonly used in the art may be used, such as an acid addition salt formed by a pharmaceutically acceptable free acid or a base addition salt formed by a free base. Specifically, the pharmaceutically acceptable salt may be an acid addition salt selected from the group consisting of acetate salt, adipate salt, aspartate salt, benzoate salt, besylate salt, bicarbonate/carbonate salt, bisulphate/sulphate salt, borate salt, camsylate salt, citrate salt, cyclamate salt, edisylate salt, esylate salt, formate salt, fumarate salt, gluceptate salt, gluconate salt, glucuronate salt, hexafluorophosphate salt, hibenzate salt, hydrochloride/chloride salt, hydrobromide/bromide salt, hydroiodide/iodide salt, isethionate salt, lactate salt, malate salt, maleate salt, malonate salt, mesylate salt, methylsulphate salt, naphthylate salt, 2-napsylate, nicotinate, nitrate, orotate, oxalate, palmitate salt, pamoate salt, phosphate/hydrogen phosphate/dihydrogen phosphate salt, pyroglutamate salt, saccharate salt, stearate salt, succinate salt, tannate salt, tartrate salt, tosylate salt, trifluoroacetate salt and xinofoate salt.

Alternatively, specific examples of the pharmaceutically acceptable salt include alkali metal salts such as lithium salt, sodium salt and potassium salt; alkaline earth metal salts such as calcium salt and magnesium salt; ammonium salt; and organic base salts such as triethylamine salt, diisopropylamine salt, or cyclohexylamine salt. The pharmaceutically acceptable salt may be specifically an alkali metal salt, more specifically a sodium salt.

However, the pharmaceutically acceptable salt is not limited to those listed above, and any conventional salt may be used without limitation as long as it is a salt that may exhibit the pharmacological activity of tegoprazan. Specifically, the pharmaceutically acceptable salt may be a tegoprazan pidolate salt or a tegoprazan malate salt.

The pharmaceutical composition for preventing peptic ulcer and/or recurrence thereof containing tegoprazan, an optical isomer thereof, a pharmaceutically acceptable salt thereof, a hydrate or solvate thereof, or a mixture thereof according to the present invention may further contain pharmaceutically acceptable additives or commonly used suitable carriers, excipients, disintegrants, binders, lubricants or diluents.

As used herein, the term “pharmaceutically acceptable additives” may include carriers, excipients, disintegrants, binders, lubricants or diluents that do not irritate an organism and do not impair the biological activity and properties of the compound to be injected. The kinds of additives that may be used in the present invention are not particularly limited, and any pharmaceutically acceptable additives commonly used in the art may be used. Non-limiting examples of the additives include mannitol, microcrystalline cellulose, croscarmellose sodium, hydroxypropyl cellulose, colloidal silicon dioxide, magnesium stearate, or mixtures thereof. In addition, if necessary, other conventional additives such as antioxidants, buffers and/or bacteriostatic agents may be added and used.

The pharmaceutical composition for preventing peptic ulcer and/or recurrence thereof containing tegoprazan or a pharmaceutically acceptable salt thereof according to the present invention may be formulated for oral administration. Specifically, the pharmaceutical composition may be formulated as a tablet or a capsule. The tablet or the capsule may be the same as described above, unless there are contradictions.

The present invention provides a pharmaceutical composition for co-administration for preventing occurrence and/or recurrence of peptic ulcer, containing: a non-steroidal anti-inflammatory drug; and tegoprazan or a pharmaceutically acceptable salt thereof,

wherein the pharmaceutical composition may effectively and safely prevent peptic ulcer or recurrence thereof caused by administration of the non-steroidal anti-inflammatory drug, for a long period of time without side effects.

In examples of the present invention, the pharmaceutical composition for co-administration may contain tegoprazan, an optical isomer thereof, a pharmaceutically acceptable salt thereof, a hydrate or solvate thereof, or a mixture thereof in an amount of 25 mg to 50 mg as tegoprazan (a free base form). Specifically, the pharmaceutical composition for co-administration may contain tegoprazan, an optical isomer thereof, a pharmaceutically acceptable salt thereof, a hydrate or solvate thereof, or a mixture thereof in an amount of 25 mg or 50 mg as tegoprazan (a free base form).

In examples of the present invention, the non-steroidal anti-inflammatory drug and tegoprazan or a pharmaceutically acceptable salt thereof may be formulated separately. In this case, a unit dosage form of the non-steroidal anti-inflammatory drug and a unit dosage form of tegoprazan or a pharmaceutically acceptable salt thereof may be administered to a subject simultaneously, sequentially, or separately with a time lag of less than 1 day. The subject may be a subject who experienced peptic ulcer but is in a state in which peptic ulcer has been treated before administration of the pharmaceutical composition for co-administration. In examples of the present invention, the non-steroidal anti-inflammatory drug and tegoprazan or a pharmaceutically acceptable salt thereof may be independently formulated into a tablet or a capsule. The tablet or the capsule may be the same as described above, unless there are contradictions.

In examples of the present invention, the non-steroidal anti-inflammatory drug and tegoprazan or a pharmaceutically acceptable salt thereof may be formulated in a single unit dosage form and administered to a subject at the same time. The subject may be a subject who experienced peptic ulcer but is in a state in which peptic ulcer has been treated before administration of the pharmaceutical composition for co-administration. In examples of the present invention, the unit dosage form may be a tablet or a capsule. The tablet or the capsule may be the same as described above, unless there are contradictions.

The present invention provides a combination for preventing peptic ulcer and/or recurrence thereof, containing: a non-steroidal anti-inflammatory drug; and tegoprazan, an optical isomer thereof, a pharmaceutically acceptable salt thereof, a hydrate or solvate thereof, or a mixture thereof.

The combination may effectively and safely prevent, for a long period of time, peptic ulcer and/or recurrence thereof caused by the non-steroidal anti-inflammatory drug.

In examples of the present invention, the combination may contain a non-steroidal anti-inflammatory drug; and tegoprazan, an optical isomer thereof, a pharmaceutically acceptable salt thereof, a hydrate or solvate thereof, or a mixture thereof in an amount of 25 mg to 50 mg as tegoprazan (a free base form). Specifically, the combination may contain a non-steroidal anti-inflammatory drug; and tegoprazan, an optical isomer thereof, a pharmaceutically acceptable salt thereof, a hydrate or solvate thereof, or a mixture thereof in an amount of 25 mg or 50 mg as tegoprazan.

In examples of the present invention, the nonsteroidal anti-inflammatory drug and tegoprazan or a pharmaceutically acceptable salt thereof may be administered to the subject simultaneously, sequentially, or independently administered with a time lag of less than 1 day.

In examples of the present invention, the non-steroidal anti-inflammatory drug and tegoprazan or a pharmaceutically acceptable salt thereof may be formulated in separate unit dosage forms, or may be formulated in a single unit dosage form.

The unit dosage form, the subject and the like may be substantially the same as descried above, unless there are contradictions.

The present invention provides a method of preventing peptic ulcer by administrating to a subject a pharmaceutically effective amount of the pharmaceutical composition of the present invention, which contains tegoprazan, an optical isomer thereof, a pharmaceutically acceptable salt thereof, a hydrate or solvate thereof, or a mixture thereof.

The present invention provides a method of preventing the recurrence of peptic ulcer by administering a pharmaceutically effective amount of the pharmaceutical composition of the present invention, which contains tegoprazan, an optical isomer thereof, a pharmaceutically acceptable salt thereof, a hydrate or solvate thereof, or a mixture thereof, to a subject who experienced peptic ulcer, but whose peptic ulcer has been treated.

In the above-described methods, the pharmaceutical composition of the present invention, the subject, the peptic ulcer and the like are substantially the same as descried above, unless there are contradictions.

According to the methods of the present invention, it is possible to safely and effectively prevent peptic ulcer and/or recurrence thereof caused by administration of a non-steroidal anti-inflammatory drug, for a long period of time without side effects, despite the non-steroidal anti-inflammatory drug that is co-administered with the pharmaceutical composition.

In the present invention, the term “pharmaceutically effective amount” refers to an amount sufficient to treat a disease at a reasonable benefit/risk ratio applicable to any medical treatment. The pharmaceutically effective amount level of the pharmaceutical composition may be determined depending on factors including the patient's type, the severity of the disease, the activity of the drug, sensitivity to the drug, the duration of administration, the route of administration, excretion rate, the duration of treatment, and drugs used in combination with the composition, as well as other factors well known in the medical field. It is important to administer the composition in the minimum amount that can exhibit the maximum effect without causing side effects, in view of all the above-described factors, and this amount can be easily determined by a person skilled in the art.

In the methods for prevention according to the present invention, it is possible to effectively and safely prevent, for a long period of time, peptic ulcer from occurring or recurring due to administration of a non-steroidal anti-inflammatory drug, by administering to the subject tegoprazan, an optical isomer thereof, a pharmaceutically acceptable salt thereof, a hydrate or solvate thereof, or a mixture thereof once a day in an amount of 25 to 50 mg as tegoprazan (a free base form). Specifically, it is possible to effectively and safely prevent, for a long period of time, peptic ulcer from occurring or recurring due to administration of a non-steroidal anti-inflammatory drug, by administering to the subject tegoprazan, an optical isomer thereof, a pharmaceutically acceptable salt thereof, a hydrate or solvate thereof, or a mixture thereof once a day in an amount of 25 or 50 mg as tegoprazan (a free base form).

The present invention provides a method of preventing peptic ulcer and/or recurrence thereof by co-administering tegoprazan or a pharmaceutically acceptable salt thereof with a non-steroidal anti-inflammatory drug.

In the method of the present invention, the tegoprazan, optical isomer thereof, pharmaceutically acceptable salt thereof, hydrate or solvate thereof, or mixture thereof may be co-administered with a non-steroidal anti-inflammatory drug to a subject who experienced peptic ulcer, but whose peptic ulcer has been treated before administration of tegoprazan, or a pharmaceutically acceptable salt thereof, once a day in an amount of 25 mg to 50 mg as tegoprazan (a free base form), specifically 25 mg or 50 mg as tegoprazan.

In examples of the present invention, the nonsteroidal anti-inflammatory drug and tegoprazan or a pharmaceutically acceptable salt thereof may be administered to a subject simultaneously or sequentially, or may be independently administered with a time difference within 1 day.

In embodiments of the present invention, the nonsteroidal anti-inflammatory drug and tegoprazan or a pharmaceutically acceptable salt thereof may be administered to the subject simultaneously, sequentially, or independently with a time lag of less than 1 day.

In examples of the present invention, the non-steroidal anti-inflammatory drug and tegoprazan or a pharmaceutically acceptable salt thereof may be formulated in separate unit dosage forms, or may be formulated in a single unit dosage form.

The pharmaceutical composition, the unit dosage form, the subject, the peptic ulcer and the like may be substantially the same as those described above, unless there are contradictions.

The present invention provides a use of the pharmaceutical composition of the present invention for preventing peptic ulcer, the pharmaceutical composition containing tegoprazan, an optical isomer thereof, a pharmaceutically acceptable salt thereof, a hydrate or solvate thereof, or a mixture thereof.

The present invention provides a use of the pharmaceutical composition of the present invention for preventing the recurrence of peptic ulcer by administration to a subject who experienced peptic ulcer, but has been treated, the pharmaceutical composition containing tegoprazan, an optical isomer thereof, a pharmaceutically acceptable salt thereof, a hydrate or solvate thereof, or a mixture thereof.

In the use of the present invention, the pharmaceutical composition contains tegoprazan, an optical isomer thereof, a pharmaceutically acceptable salt thereof, a hydrate or solvate thereof, or a mixture thereof in an amount of 25 to 50 mg as tegoprazan (a free base form), specifically 25 mg or 50 mg as tegoprazan, and may be administered once a day, thus effectively and safely preventing, for a long period of time, peptic ulcer from occurring or recurring due to administration of a non-steroidal anti-inflammatory drug.

In examples of the present invention, the nonsteroidal anti-inflammatory drug and tegoprazan or a pharmaceutically acceptable salt thereof may be administered to a subject simultaneously, sequentially, or independently with a time lag of less than 1 day.

In examples of the present invention, the non-steroidal anti-inflammatory drug and tegoprazan or a pharmaceutically acceptable salt thereof may be formulated in separate unit dosage forms, or may be formulated in a single unit dosage form.

The pharmaceutical composition, the unit dosage form, the subject, the peptic ulcer and the like may be substantially the same as those described above, unless there are contradictions.

The present invention provides a use of the pharmaceutical composition for preventing peptic ulcer and/or recurrence thereof, the pharmaceutical composition containing a non-steroidal anti-inflammatory drug and tegoprazan, an optical isomer thereof, a pharmaceutically acceptable salt thereof, a hydrate or solvate thereof, or a mixture thereof.

In the use of the present invention, the pharmaceutical composition contains tegoprazan, an optical isomer thereof, a pharmaceutically acceptable salt thereof, a hydrate or solvate thereof, or a mixture thereof in an amount of 25 to 50 mg as tegoprazan (a free base form), specifically 25 mg or 50 mg as tegoprazan, and may be administered once a day, thus effectively and safely preventing, for a long period of time, peptic ulcer from occurring or recurring due to administration of the non-steroidal anti-inflammatory drug.

In examples of the present invention, the nonsteroidal anti-inflammatory drug and tegoprazan or a pharmaceutically acceptable salt thereof may be administered to a subject simultaneously, sequentially, or independently with a time lag of less than 1 day.

In examples of the present invention, the non-steroidal anti-inflammatory drug and tegoprazan or a pharmaceutically acceptable salt thereof may be formulated in separate unit dosage forms, or may be formulated in a single unit dosage form.

The pharmaceutical composition, the unit dosage form, the subject, the peptic ulcer and the like may be substantially the same as those described above, unless there are contradictions.

The features mentioned in the pharmaceutical composition of the present invention may be equally applied to the pharmaceutical composition for co-administration, the combination, the method for prevention and the use, unless there are contradictions.

### Advantageous Effects

The present invention relates to a pharmaceutical composition for preventing peptic ulcer and/or recurrence thereof, containing tegoprazan, an optical isomer thereof, a pharmaceutically acceptable salt thereof, a hydrate or solvate thereof, or a mixture thereof. The pharmaceutical composition may effectively and safely prevent, for a long period of time, peptic ulcer and/or recurrence thereof caused by administration of a nonsteroidal anti-inflammatory drug.

### DESCRIPTION OF DRAWINGS

FIG. 1 schematically shows a clinical trial procedure performed using a pharmaceutical composition of the present invention.

### MODE FOR INVENTION

Hereinafter, the present invention will be described in more detail with reference to examples. However, these examples serve to describe the present invention in more detail, and the scope of the present invention is not limited by these examples.

### Preparation Example 1: Preparation of Pharmaceutical Formulation (1)-25 mg Tegoprazan Tablet

A dosage form was prepared to contain 25 mg of (S)-4-[(5,7-difluoro-3,4-dihydro-2H-chromene-4-yl)oxy]-N,N,2-trimethyl-1H-benzimidazole-6-carboxamide as an active ingredient. The active ingredient was mixed with mannitol, microcrystalline cellulose, and croscarmellose sodium. The fillers were contained in an amount of 1 to 99 wt % (mannitol: 25 mg, and microcrystalline cellulose: 40 mg) based on the total weight of the final dosage form, and the disintegrant was contained in an amount of 1 to 20 wt % (croscarmellose sodium: 5 mg) based on the total weight of the final dosage form.

The mixture was granulated by adding a binder solution containing hydroxypropyl cellulose and purified water, and the binder was used in an amount of 4 to 40 wt % (4 mg hydroxypropyl cellulose) based on the weight of the active ingredient.

The granules were dried and then sized, and microcrystalline cellulose, croscarmellose sodium, colloidal silicon dioxide and magnesium stearate were added to the granules and mixed together.

The diluent was used in an amount of 1 to 10 wt % (1 mg colloidal silicon dioxide) based on the total weight of the final dosage form, and the lubricant was used in an amount of 1 to 10 wt % (1 mg magnesium stearate) based on the total weight of the final dosage form. The resulting mixture was compressed into a tablet.

The tablet was coated with a film coating agent. The coating agent was used in an amount of 2 to 6 wt % (3 mg) based on the total weight of the final dosage form.

### Preparation Example 2: Placebo for 25 mg Tegoprazan

A placebo for 25 mg tegoprazan was prepared in the same manner as in Preparation Example 1, except that the active ingredient tegoprazan was not used.

### Preparation Example 3: Preparation of 15 mg Lansoprazole Formulation

For a lansoprazole formulation, 15 mg Lanston capsule (15 mg lansoprazole) purchased from Jeil Pharmaceutical Co., Ltd. was used.

Preparation Example 4: Placebo for 15 mg Lansoprazole A placebo for 15 mg lansoprazole was prepared in the same manner as the Lanston capsule preparation method, except that the active ingredient lansoprazole was not used and a pharmaceutically acceptable excipient was added.

### Example 1

1. Selection of Subjects

To evaluate the safety and gastroduodenal ulcer preventive effect of tegoprazan in patients who need to continuously take nonsteroidal anti-inflammatory drugs (NSAIDs), a randomized, double-blind, active-controlled clinical trial was designed.

Inclusion Criteria

Unless otherwise specified, trial subjects included in this clinical trial satisfied the following selection criteria.

(1) Those who are 20 years of age or older as of the date of obtaining written consent;

(2) Those who are 60 years of age or older or 20 years of age or older and who have a history of gastroduodenal ulcer (gastric ulcer and/or duodenal ulcer) at the screening visit
the history of gastroduodenal ulcer refers to a case where a scar is confirmed by upper gastrointestinal tract endoscopy performed at the time of screening, or where the diagnosis is confirmed by endoscopy findings in the past medical records;

(3) those who need to take non-steroidal anti-inflammatory drugs (non-selective NSAIDs, COX-2 selective inhibitors) continuously for 24 weeks or more after randomization without changing the type and dose of the drug, due to musculoskeletal diseases (e.g., osteoarthritis, rheumatoid arthritis, etc.)
those who take indomethacin, naproxen, aceclofenac, diclofenac, piroxicam, meloxicam, ibuprofen, loxoprofen, celecoxib, etc. are included, and those who take acetaminophen and aspirin are excluded (however, those who take low doses of aspirin are not excluded);

(4) those who can understand and follow instructions and can participate throughout the entire clinical trial period;

(5) those who voluntarily decide to participate and consent in writing to participate in the clinical trial; and

(6) those who are medically incapable of conceiving or has agreed to use a medically valid contraceptive method during the clinical trial period.

Exclusion Criteria

Those who met any of the following criteria were excluded from this clinical trial.

(1) Those who were confirmed to have gastric duodenal ulcers corresponding to the active phase (A1, A2) and healing phase (H1, H2) according to the Sakita-Miwa classification in the upper gastrointestinal endoscopy performed at the time of screening;

(2) those who were confirmed to have gastrointestinal bleeding, perforation, esophageal stenosis, ulcer stenosis, pyloric stenosis, esophageal gastric varicose veins, long segment Barrett esophagus (LSBE) exceeding 3 cm, all grades of dysplastic changes in the esophagus, and malignant tumors in the upper gastrointestinal endoscopy performed at the time of screening;

(3) those who have symptoms such as odynophagia, pain, dysphagia, bleeding, weight loss, anemia, and bloody stools (except for hemorrhoids), which are “warning symptoms” that can indicate malignant diseases of the gastrointestinal tract (however, among those who show warning symptoms, those whose tumors were negatively diagnosed by endoscopy may be included);

(4) those who previously underwent serious surgery that can affect gastric acid secretion, such as upper gastrointestinal duct resection, gastric acid secretion suppression, gastric mucosal resection, etc.

(except simple perforation surgery, metacarpophalectomy, cholecystectomy, and benign tumor resection using laparoscopy);

(5) those who were diagnosed with or have a medical history of Zolinger-Ellison syndrome or other gastric acid secretion disorders;

(6) patients with severe, uncontrolled hypertension (siDBP≥110 mmHg or siSBP≥180 mmHg at the time of screening);

(7) patients who have severe heart failure, congestive heart failure (NYHA II to IV), ischemic heart disease (unstable angina, myocardial infarction), peripheral arterial disease, or cerebrovascular disease, or underwent coronary artery bypass graft (CABG) treatment, and who were judged to be unable to administer nonsteroidal anti-inflammatory drugs;

(8) those with severe blood abnormalities or blood clotting disorders;

(9) those with inflammatory diseases (inflammatory bowel disease such as Crohn's disease or ulcerative colitis, pancreatitis, etc.);

(10) those who were confirmed to be positive for H. pylori in a test conducted at the time of screening;

(11) those who need to continuously take corticosteroids, antithrombotic agents, and anticoagulants during the trial period

(however, it is permitted to take aspirin at a low dose (100 mg or less per day), which was used for the purpose of preventing cardiovascular disease before participation in the clinical trial);

(12) patients with clinically significant liver impairment (AST, ALT, ALP, Y-CT, and total bilirubin levels are more than twice the upper limit of the normal range for each laboratory);

(13) patients with clinically significant renal impairment (creatinine level in blood is more than twice the upper limit of the normal range for each laboratory);

(14) those with clinically significant abnormal ECG;

(15) those with a history of malignant tumors within the recent 5 years
those who were diagnosed with complete remission (CR, pCR) of the tumor and have passed 5 years or more without recurrence from the date of diagnosis, and those who have had more than 3 years without recurrence after the tumor has been completely removed through endoscopic gastric mucosal resection (mucosal resection (EMR), submucosal dissection (ESD)) may be included;

(16) patients who have hypersensitivity reactions and medical history to medicament for clinical trial and benzimidazoles or non-steroidal anti-inflammatory drugs components scheduled to be used in combination during the clinical trial;

(17) patients with a history of asthma, rhinitis, nasal polyps, angioedema, urticaria, or allergic responses to aspirin or other nonsteroidal anti-inflammatory drugs (including COX-2 inhibitors);

(18) patients taking drugs containing HIV protease inhibitors (atazanavir or nelfinavir) or rilpivirine;

(19) subjects who are scheduled for surgery that requires hospitalization or require surgical treatment during participation in the clinical trial;

(20) those who participated in other clinical trial and administered the drug for the other clinical trial within 4 weeks based on a start date of administration of a drug for this clinical trial
clinical trial subjects who have participated in or are participating in non-interventional studies (non-interventional studies such as observational studies, questionnaire survey, etc.) that the investigator has determined that there will be no impact on the efficacy and safety evaluation of this clinical trial can participate in this clinical trial
those who have been dropped out of screening without administering clinical trial drugs after writing consent for participation in other clinical trials can participate in this clinical trial;

(21) pregnant or lactating women;

(22) patients with a history of alcohol abuse;

(23) in addition to the above, those who have clinically significant findings deemed inappropriate for this test as a result of medical judgment by.

2. Clinical Trial Design

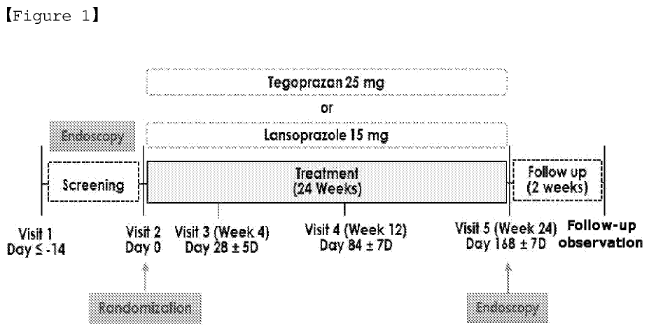

The clinical trial was carried out by a double-blind, randomized, active drug control, multicenter clinical trial method, and the clinical trial procedure may be schematically illustrated in FIG. 1.

Subjects who visited for the clinical trial were assigned a screening number on the first day of visit (visit 1) in the order agreed upon, and subjected to a screening test (endoscopy), and trial subjects were selected through past medical history and gastroscopy. The selected clinical trial subjects were randomly assigned (visit 2) and classified into group 2 (195 people for each group).

Drugs were administered to each group for 24 weeks and endoscopy was performed.

3. Dosage and Method of Administration

As shown in Table 1, one tablet and one capsule were administered to the two classified groups.

TABLE 1

| | |
|---|---|
| Test group | Preparation Example 1 + Preparation Example 4 |
| Control group | Preparation Example 2 + Preparation Example 3 |

As shown in Table 1 above, 25 mg-tegoprazan tablet (Preparation Example 1)/15-mg lansoprazole placebo (Preparation Example 4) were orally administered to the test group, and 25-mg tegoprazan placebo (Preparation Example 2)/15-mg lansoprazole capsule (Preparation Example 3) were orally administered to the control group.

Subjects randomly assigned to each group were administered nonsteroidal anti-inflammatory drugs according to the dosage and regimen prescribed for each subject during the clinical trial period, and diary records for the clinical trial subjects were also prepared from the day of start of administration. Subjects randomly assigned to each group were administered the prescribed clinical trial drug once a day at a certain time starting from the next day (day 1) after prescription. The trial subject diaries were prepared from the day when administration of the clinical trial drug started.

On the day of the visit for upper gastrointestinal endoscopy, visit without taking nonsteroidal anti-inflammatory drugs and the clinical trial drug.

At week 4 (visit 3), week 12 (visit 4) and week 24 (visit 5), each subject visited on an empty stomach without taking the clinical trial drug. On visit 3 and visit 4, after the scheduled examination, a newly prescribed clinical trial drug and a non-steroidal anti-inflammatory drug that had been administered (or newly prescribed) were administered.

Visit at 24 weeks of medication administration to check whether an ulcer has occurred on upper gastrointestinal endoscopy, and adverse reactions, clinical laboratory tests (hematology, blood chemistry, blood coagulation, urine tests, etc.), vitality tests (blood pressure in sitting position, heart rate, body temperature), electrocardiogram, physical examination and symptom evaluation, etc. were performed. The clinical trial subjects entered the safety f/u (follow-up period) after taking the clinical trial drug for up to 24 weeks, and the follow-up observation (phone call or visit) was carried out 2 weeks after the last administration.

4. Efficacy Evaluation

A. Primary Efficacy Evaluation

Percentage (%) of subjects who developed gastric ulcer and/or duodenal ulcer after 24 weeks

: percentage (%) of subjects who developed gastric ulcer and/or duodenal ulcer as a result of upper gastrointestinal endoscopy after 24 weeks of administration of clinical trial drug

=(number of subjects who developed gastric ulcer and/or duodenal ulcer after 24 weeks of administration of clinical trial drug/number of subjects who received upper gastrointestinal endoscopy after 24 weeks of administration of clinical trial drug)×100

When a subject was diagnosed with a gastric ulcer and/or duodenal ulcer corresponding to the active stage (A1, A2) and healing stage (H1, H2) according to the Sakita-Miwa classification as a result of endoscopy, the subject was considered to have developed gastric ulcer and/or duodenal ulcer.

TABLE 2
Sakita-Miwa Classification

| Stage | Class | Manifestation |
|---|---|---|
| Active | A1 | The surrounding mucosa is edematously swollen and |
| stage | | no regenerating epithelium is seen endoscopically. |
| | A2 | The surrounding edema has decreased, the ulcer |
| | | margin is clear, and a slight amount of |
| | | regenerating epithelium is seen in the ulcer |
| | | margin. Red halo of the marginal zone and white |
| | | slough circle of the ulcer margin are seen |
| | | frequently. Usually, converging mucosal folds can |
| | | be followed right up to the ulcer margin. |
| Healing | H1 | The white coating is becoming thin and the |
| stage | | regenerating epithelium is extending into the |
| | | ulcer base. The gradient between the ulcer margin |
| | | and the ulcer floor is becoming flat. The ulcer |
| | | crater is still evident and the margin of the ulcer |
| | | is sharp. The diameter of the mucosal defect is |
| | | about one-half to two-thirds that of A1. |
| | H2 | The defect is smaller than in H1 and the |
| | | regenerating epithelium covers most of the ulcer |
| | | floor. The area of white coating is about a quarter |
| | | to one-third that of A1. |
| Scarring | S1, S2 | The regenerating epithelium completely covers the |
| stage | | floor of ulcer. The white coating has disappeared. |
| | | Initially, the regenerating region is markedly |
| | | red. Upon close observation many capillaries can |
| | | be seen. This is called ″red scar″ (S1) . In several |
| | | months to a few years, the redness is reduced to |
| | | the color of the surrounding mucosa. This is |
| | | called ″white scar″ (S2). |

B. Secondary Efficacy Evaluation Variables

Percentage (%) of subjects without gastrointestinal symptoms associated with administration of NSAIDs after 4, 12, and 24 weeks

(1) Percentage (%) of subjects without heartburn

(2) Percentage (%) of subjects without gastric acid reflux

(3) Percentage (%) of subjects without epigastric pain or discomfort

5. Additional Evaluation Variables

Changes in blood hemoglobin (Hb) concentration after 4, 12, and 24 weeks compared to baseline

The baseline and the mean, standard deviation, median, minimum and maximum values of the blood hemoglobin (Hb) concentration were checked 4 weeks, 12 weeks, and 24 weeks after administration of the clinical trial drug.

6. Subgroup Analysis and Evaluation

A. The incidence (%) of gastric duodenal ulcer according to the history of gastric duodenal ulcer

The incidence of gastric duodenal ulcer depending on the history of gastric duodenal ulcer was checked 24 weeks after administration of the clinical trial drug.

B. The incidence (%) of gastric duodenal ulcers depending on non-selective NSAIDs and COX-2 selective inhibitors

The incidence of gastric duodenal ulcer depending on administration of non-selective NSAIDs and COX-2 selective inhibitors was checked 24 weeks after administration of the clinical trial drug.

C. The incidence (%) of gastric duodenal ulcer depending on co-administration of low-dose aspirin

The incidence of gastric duodenal ulcer depending on co-administration of low-dose aspirin was checked 24 weeks after administration of the clinical trial drug.

7. Safety Evaluation

Adverse reactions, clinical laboratory tests (hematology test, blood chemistry test, blood coagulation test, and urine test), vital signs (blood pressure in sitting position, heart rate, and body temperature), electrocardiogram, and physical examination

A. Adverse Reactions

The number of clinical trial subjects who developed adverse reactions, adverse drug reactions, serious adverse reactions, and serious adverse drug reactions, and incidence rate of adverse reactions, adverse drug reactions, serious adverse reactions, and serious adverse drug reactions were checked.

B. Clinical Laboratory Tests

For continuous data on the items of hematology, blood chemistry, blood coagulation and urine tests, the mean, standard deviation, maximum and minimum values before administration of the clinical trial drug and at the last visit during the administration period were checked. For categorical data, the shift table was checked by frequency and percentage.

C. Vital Signs

For vital signs (systolic blood pressure in sitting position, diastolic blood pressure in sitting position, heart rate, and body temperature), the mean, standard deviation, minimum and maximum values before administration of the clinical trial drug and at the last visit during the administration period were checked.

D. Electrocardiography

For ECG, the results of electrocardiography before administration of the clinical trial drug and at the last visit during the administration period were expressed as frequency and percentage on the shift table and checked.

E. Physical Examination

For physical examination, the results of each physical examination item before administration of the clinical trial drug and at the last visit during the administration period were expressed as frequency and ratio on the shift table and checked.

### Example 2

To confirm the effect of tegoprazan on peptic ulcers induced by nonsteroidal anti-inflammatory drugs (NSAIDs), the anti-ulcer activity of tegoprazan in an animal model of acute peptic ulcer induced by naproxen, which is a nonsteroidal anti-inflammatory drug, was evaluated, and the anti-ulcer effect of tegoprazan was compared with the anti-ulcer effect of esomeprazole.

Specifically, each of tegoprazan (0.1 to 10 mg/kg) and esomeprazole (0.3 to 30 mg/kg) was orally administered to each SD rat that has fasted for 72 hours. After 30 minutes, naproxen (30 mg/kg) was orally administered to each rat three times at intervals of 2 hours. Four hours after the last administration of naproxen, the stomach of each rat was collected.

As a result, it can be confirmed that tegoprazan exhibited excellent anti-ulcer activity in the naproxen-induced peptic ulcer model, and the efficacy of tegoprazan (ED₅₀, 0.123 mg/kg) was better than that of esomeprazole (ED₅₀, 4.116 mg/kg).

Therefore, it can be confirmed that tegoprazan can prevent peptic ulcer caused by non-steroidal anti-inflammatory drugs.

### Example 3

To evaluate the pharmacokinetic interactions between tegoprazan and nonsteroidal anti-inflammatory drugs, pharmacokinetic parameters were evaluated when tegoprazan was repeatedly co-administered with the nonsteroidal anti-inflammatory drugs naproxen, aceclofenac and celecoxib in randomized, open, repeated administration, 6-group, and phase-3 crossover trials.

[Cohort 1]

The pharmacokinetic interactions of tegoprazan and naproxen, when administered alone or in combination, were evaluated in healthy adult males.

[Cohort 2]

The pharmacokinetic interactions of tegoprazan and aceclofenac when administered alone or in combination were evaluated in healthy adult males.

[Cohort 3]

The pharmacokinetic interactions of tegoprazan and celecoxib when administered alone or in combination were evaluated in healthy adult males.

The results of the evaluation are shown in Tables 3 to 8 below.

In Tables 3 to 8 below, Treatment A represents a group to which 50 mg tegoprazan alone was administered once a day for 7 days; Treatment B represents a group to which 500 mg naproxen alone was administered twice a day for 7 days; Treatment C represents to a group to which 50 mg tegoprazan (once a day) was co-administered with 500 mg naproxen (twice a day) for 7 days; Treatment D represents a group to which 100 mg aceclofenac alone was administered twice a day for 7 days; Treatment E represents a group to which 50 mg tegoprazan (once a day) was co-administered with 100 mg aceclofenac (twice a day) for 7 days; Treatment F represents a group to which 200 mg celecoxib alone was administered twice a day for 7 days; and Treatment G represents 50 mg tegoprazan (once a day) was co-administered with 200 mg celecoxib (twice a day) for 7 days.

(1) Tegoprazan and Naproxen

Tables 3 and 4 below show the pharmacokinetic parameters obtained when each of tegoprazan and naproxen was administered or tegoprazan and naproxen co-administered.

TABLE 3
Tegoprazan

| | | | Geometric LS Mean Ratio | |
|---|---|---|---|---|
| | Geometric LS Mean | | (Treatment C/Treatment A) | |
| | Treatment | Treatment | | 90% |
| Pharmacokinetic | C | A | Point | Confidence |
| Parameter (unit) | (N = 17) | (N = 17) | Estimate | Interval |
| AUC_{τ} (h*ng/mL) | 2674.97 | 2647.22 | 1.0105 | 0.9081-1.1244 |
| C_{ss,max} (ng/mL) | 505.59 | 512.79 | 0.9859 | 0.8281-1.1739 |

TABLE 4
Naproxen

| | | | Geometric LS Mean Ratio | |
|---|---|---|---|---|
| | Geometric LS Mean | | (Treatment C/Treatment B) | |
| | Treatment | Treatment | | 90% |
| Pharmacokinetic | C | B | Point | Confidence |
| Parameter (unit) | (N = 17) | (N = 17) | Estimate | Interval |
| AUC_{τ} (h*ng/mL) | 743.48 | 742.66 | 1.0011 | 0.97217-1.0314 |
| C_{ss,max} (ng/mL) | 95.60 | 92.34 | 1.0353 | 0.9867-1.0862 |

Referring to Tables 3 and 4, as a result of comparing the co-administration of tegoprazan and naproxen with the administration thereof alone, it can be confirmed that the co-administration does not affect the pharmacokinetic properties of tegoprazan and naproxen.

(2) Tegoprazan and Aceclofenac

Tables 5 and 6 below show the pharmacokinetic parameters obtained when each of tegoprazan and aceclofenac was administered or tegoprazan and aceclofenac co-administered.

TABLE 5
Tegoprazan

| | | | Geometric LS Mean Ratio | |
|---|---|---|---|---|
| | Geometric LS Mean | | (Treatment E/Treatment A) | |
| | Treatment | Treatment | | 90% |
| Pharmacokinetic | E | A | Point | Confidence |
| Parameter (unit) | (N = 16) | (N = 16) | Estimate | Interval |
| AUC_{τ} (h*ng/mL) | 2751.73 | 2679.62 | 1.0269 | 0.9296-1.1344 |
| C_{ss,max} (ng/mL) | 529.98 | 562.13 | 0.9428 | 0.8565-1.0378 |

TABLE 6
Aceclofenac

| | | | Geometric LS Mean Ratio | |
|---|---|---|---|---|
| | Geometric LS Mean | | (Treatment E/Treatment D) | |
| | Treatment | Treatment | | 90% |
| Pharmacokinetic | E | D | Point | Confidence |
| Parameter (unit) | (N = 16) | (N = 16) | Estimate | Interval |
| AUC_{τ} (h*ug/mL) | 21.22 | 20.06 | 1.0578 | 1.0009-1.1180 |
| C_{ss,max} (ug/mL) | 10.91 | 8.30 | 1.3149 | 1.0834-1.5959 |

Referring to Tables 5 and 6, as a result of comparing the co-administration of tegoprazan and aceclofenac with the administration thereof alone, it can be confirmed that the co-administration does not affect the pharmacokinetic properties of tegoprazan and aceclofenac.

In addition, regarding aceclofenac, the results of AUC, which reflects the total amount (extent) of the drug that is absorbed into the body and reaches the systemic circulation, between co-administration and single administration are similar, and therefore, it is not predicted to have a clinically significant change.

Thus, as a result of comparing the co-administration of tegoprazan and aceclofenac with the administration thereof alone, it can be confirmed that the co-administration does not affect the pharmacokinetic properties of tegoprazan and aceclofenac.

(3) Tegoprazan and Celecoxib

Tables 7 and 8 below show the pharmacokinetic parameters obtained when each of tegoprazan and celecoxib was administered or tegoprazan and celecoxib co-administered.

TABLE 7
Tegoprazan

| | | | Geometric LS Mean Ratio | |
|---|---|---|---|---|
| | Geometric LS Mean | | (Treatment G/Treatment A) | |
| | Treatment | Treatment | | 90% |
| Pharmacokinetic | G | A | Point | Confidence |
| Parameter (unit) | (N = 13) | (N = 13) | Estimate | Interval |
| AUC_{τ} (h*ng/mL) | 3023.94 | 2990.68 | 1.0111 | 0.8641-1.1832 |
| C_{ss,max} (ng/mL) | 676.24 | 663.83 | 1.0187 | 0.8699-1.1930 |

TABLE 8
Celecoxib

| | | | Geometric LS Mean Ratio | |
|---|---|---|---|---|
| | Geometric LS Mean | | (Treatment G/Treatment F) | |
| | Treatment | Treatment | | 90% |
| Pharmacokinetic | G | F | Point | Confidence |
| Parameter (unit) | (N = 13) | (N = 13) | Estimate | Interval |
| AUC_{τ} (h*ug/mL) | 8445.68 | 7801.65 | 1.0826 | 0.9618-1.2185 |
| C_{ss,max} (ug/mL) | 1346.87 | 1140.31 | 1.1811 | 0.9743-1.4319 |

Referring to Tables 7 and 8, as a result of comparing the co-administration of tegoprazan and celecoxib with the administration thereof alone, it can be confirmed that the co-administration does not affect the pharmacokinetic properties of tegoprazan and celecoxib.

In addition, regarding celecoxib, the results of AUC, which reflects the total amount (extent) of the drug that is absorbed into the body and reaches the systemic circulation, between co-administration and single administration are similar, and therefore, it is not predicted to have a clinically significant change between co-administration and single administration.

Thus, as a result of comparing the co-administration of tegoprazan and celecoxib with the administration thereof alone, it can be confirmed that the co-administration does not affect the pharmacokinetic properties of tegoprazan and celecoxib.

1. A pharmaceutical composition for preventing peptic ulcer, the pharmaceutical composition comprising tegoprazan which is a compound represented by the following Formula 1, an optical isomer thereof, a pharmaceutically acceptable salt thereof, a hydrate or solvate thereof, or a mixture thereof,
wherein the peptic ulcer is caused by administration of a non-steroidal anti-inflammatory drug (NSAID):

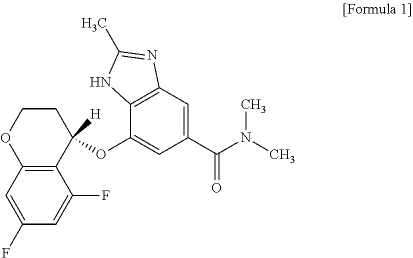 [SMILES: Cc1nc2cc(C(=O)N(C)C)cc(O[C@H]3CCOc4cc(F)cc(F)c43)c2[nH]1]

2. The pharmaceutical composition of claim 1, the pharmaceutical composition comprises the tegoprazan, optical isomer thereof, pharmaceutically acceptable salt thereof, hydrate or solvate thereof, or mixture thereof in an amount of 25 mg to 50 mg as tegoprazan.

3. The pharmaceutical composition of claim 1, the pharmaceutical composition comprises the tegoprazan, optical isomer thereof, pharmaceutically acceptable salt thereof, hydrate or solvate thereof, or mixture thereof in an amount of 25 mg as tegoprazan.

4. The pharmaceutical composition of claim 2, the pharmaceutical composition is administered to a subject who experienced peptic ulcer before administration of the pharmaceutical composition.

5. The pharmaceutical composition of claim 4, wherein the subject is a person whose peptic ulcer has been treated before administration of the pharmaceutical composition.

6. The pharmaceutical composition of claim 5, wherein the subject is a person who takes the non-steroidal anti-inflammatory drug.

7. The pharmaceutical composition of claim 2, the pharmaceutical composition is for preventing recurrence of the peptic ulcer.

8. The pharmaceutical composition of claim 1, the pharmaceutical composition further comprises a non-steroidal anti-inflammatory drug.

9. The pharmaceutical composition of claim 1, the pharmaceutical composition is co-administered with a non-steroidal anti-inflammatory drug.

10. The pharmaceutical composition of claim 8 or 9, wherein the non-steroidal anti-inflammatory drug is one or more selected from the group consisting of non-selective NSAIDs and COX-2 selective inhibitors.

11. The pharmaceutical composition of claim 8 or 9, wherein the non-steroidal anti-inflammatory drug is one or more selected from the group consisting of salicylates, propionic acid derivatives, acetic acid derivatives, enolic acid (oxicam) derivatives, anthranilic acid derivative (fenamates), selective Cox-2 inhibitors (coxibs), sulfonanilides and clonixin.

12. The pharmaceutical composition of claim 8 or 9, wherein the non-steroidal anti-inflammatory drug is one or more selected from the group consisting of propionic acid derivatives, acetic acid derivatives and selective Cox-2 inhibitor (coxibs).

13. The pharmaceutical composition of claim 12, wherein the non-steroidal anti-inflammatory drug is one or more selected from the group consisting of naproxen, aceclofenac and celecoxib.

14. The pharmaceutical composition of claim 2, the pharmaceutical composition is administered once a day.

15. The pharmaceutical composition of claim 2, the pharmaceutical composition is formulated in a unit dosage form.

16. The pharmaceutical composition of claim 8 or 9, wherein the pharmaceutical composition and the non-steroidal anti-inflammatory drug are each independently formulated in separate unit dosage forms or in a single unit dosage form.

17. A method for preventing peptic ulcer caused by administration of a non-steroidal anti-inflammatory drug (NSAID), the method comprising administering to a subject an effective amount of the pharmaceutical composition according to any one of claims 1 to 16.

18. Use of the pharmaceutical composition according to any one of claims 1 to 16 for preventing peptic ulcer caused by administration of a non-steroidal anti-inflammatory drug (NSAID).

19. Use of the pharmaceutical composition according to any one of claims 1 to 16 in the manufacture of a medicament for preventing peptic ulcer caused by administration of a non-steroidal anti-inflammatory drug (NSAID).
